(19) United States
(12) Patent Application Publication
(10) Pub. No.: US 2019/0374378 A1
(43) Pub. Date: Dec. 12, 2019

(54) DEVICES AND METHODS FOR WEARABLES

(71) Applicant: Aladdin Dreamer, Inc., Paradise Valley, AZ (US)

(72) Inventors: Craig WEISS, Paradise Valley, AZ (US); Alean DANIEL, Albany, CA (US); Stephen KROTSENG, San Francisco, CA (US); Erik ASKIN, San Francisco, CA (US); Gadi AMIT, San Mateo, CA (US)

(73) Assignee: Aladdin Dreamer, Inc., Paradise Valley, AZ (US)

(21) Appl. No.: 16/433,465

(22) Filed: Jun. 6, 2019

Related U.S. Application Data
(60) Provisional application No. 62/681,996, filed on Jun. 7, 2018.

(51) Int. Cl. A61F 7/02; A61B 5/0478; A61B 5/01; A61B 5/00

(52) U.S. Cl. CPC A61F 7/02; A61F 2007/0096; A61B 5/01; A61B 5/4266; A61B 5/6803; A61B 5/6806; A61B 5/6892; A61B 5/4836; A61B 2562/029; A61B 2562/0209; A61B 2562/066; A61F 2007/0226; A61F 2007/0228; A61F 2007/0234; A61B 5/0478

(57) ABSTRACT

A wearable device may include a first side, a second side, at least one electrode, and at least one thermocouple. The first side may be the side in contact with a subject, and the at least one electrode and the at least one thermocouple may be arranged on the first side.

### CROSS-REFERENCE TO RELATED APPLICATIONS

This application claims the benefit of priority from U.S. Provisional Patent Application No. 62/681,996, filed on Jun. 7, 2018, the entirety of which is incorporated herein by reference.

### TECHNICAL FIELD

Various aspects of the present disclosure relate generally to devices and related methods for regulating temperature. More specifically, the present disclosure relates to devices and methods for wearables coupled to a81996 subject that are capable of reading various features of a subject, detecting various stages of a subject's sleep, and/or regulating temperatures to cool or warm a subject in response to the readings, the detected sleep stages, and/or in response to a manual temperature adjustment by the subject.

### BACKGROUND

Among various environmental or surrounding conditions, temperature is one of the most significant conditions or factors that affect people on an everyday basis. From the temperatures of surrounding environments, the number of layers or type of clothes worn, to the heat conducted by a mug filled with a hot beverage, temperature has a large effect on a person's comfort level, and what they perceive to be convenient and/or favorable. The present disclosure may solve one or more of these problems or other problems in the art. The scope of the disclosure, however, is defined by the attached claims and not the ability to solve a specific problem.

### SUMMARY

Examples of the present disclosure relate to, among other things, devices and methods for regulating temperature. Each of the examples disclosed herein may include one or more of the features described in connection with any of the other disclosed examples.

In one aspect, a wearable may include a first side, a second side, at least one electrode, and at least one thermocouple. The first side may be a side that is in contact with a subject and the second side may be the side that is opposite of the first side. The at least one electrode and the at least one thermocouple may be arranged on the first side.

Examples of the wearable may additionally and/or alternatively include one or more of the following features. The thermocouple may be configured for application of the Peltier effect, thereby cooling or heating the first side of the wearable. The wearable may also include an actuator, and subject manipulation of the actuator may be configured to adjust a temperature of the first side of the wearable and/or a level of relative cooling or heating. The actuator may include at least one of a dial, a push button, or a slider. The wearable may include at least one of a band, a wrap, or an adhesive crest. The wearable may include a wrap, having at least one closure structure. The wearable may include one or more of an article of clothing, an article of bedding, or a consumer product. The wearable may also include an electrode module, and a thermocouple module. The electrode module may include the at least one electrode, and the thermocouple module may include the at least one thermocouple, and the electrode module and the thermocouple module may be arranged on the first side. The wearable may also a combination module. The combination module may include the at least one electrode and the at least one thermocouple, and the combination module may be arranged on the first side. At least one electrode may be configured to measure temperature or perspiration/humidity. At least one electrode may be configured to measure EEG brain patterns.

In another example, a method of dynamically adjusting a temperature of a wearable may include monitoring a temperature level of a subject via at least one electrode of the wearable. The method may also include, based on the monitored temperature level of the subject, applying a voltage across a thermocouple of the wearable resulting in a cooling effect or a heating effect of a first side of the wearable via the Peltier effect. The first side of the wearable may be a side in contact with the subject.

Examples of the method may additionally and/or alternatively include one or more of the following features. The method may also include comparing the monitored temperature level to a preset temperature range, and based on the monitored temperature level being above the preset temperature range, applying the voltage across the thermocouple resulting in the cooling effect of the first side of the wearable via the Peltier effect. The method may also include, based on the monitored temperature level being below the preset temperature range, applying the voltage across the thermocouple resulting in the heating effect of the first side of the wearable via the Peltier effect. Additionally, the method may include, based on the monitored temperature level being within the preset temperature range, delaying the application of the voltage across the thermocouple. The method may also include reversing a polarity of the applied voltage to at least one thermocouple resulting in the first side changing from the heating effect to the cooling effect or from the cooling effect to the heating effect. The method may additionally include monitoring EEG brain patterns of a subject via at least one additional electrode of the wearable, and based on the monitored EEG brain patterns, applying the voltage across the thermocouple of the wearable resulting in the cooling effect or the heating effect of the first side of the wearable via the Peltier effect.

In another example, a method of dynamically adjusting a temperature may include positioning at least one of a wearable or a consumer product relative to a subject. The method may also include, based on a monitored or perceived condition of the subject, applying a voltage across at least one thermocouple of the at least one of the wearable or consumer product resulting in a heating effect or a cooling effect of a first side of the at least one of the wearable or the consumer product via the Peltier effect. The first side may be a side in contact with the subject.

Examples of the method may additionally and/or alternatively include one or more of the following features. The method may include reversing a polarity of the applied voltage to at least one thermocouple resulting in the first side changing from the heating effect to the cooling effect or from the cooling effect to the heating effect. The wearable or the consumer product may include an article of clothing, footwear, an article of bedding, a head covering, earphones, a container, a temperature sleeve of a container, a safety device, a seat, or a steering wheel.

It may be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate exemplary embodiments of the present disclosure and together with the description, serve to explain the principles of the disclosure.

FIG. 1 is a chart illustrating a perceived temperature of participants utilizing a wearable device not including a Peltier effect module, over time.

FIG. 2 is a chart illustrating the perceived temperature of participants utilizing a wearable device including the Peltier effect module, over time.

FIG. 3 is a chart illustrating the perceived temperature of participants during a cycling of the Peltier effect module, over time.

FIG. 4 is a chart comparing the temperatures of participants under various conditions, over time.

FIGS. 5A-5I illustrates various embodiments of a wearable device, according to one or more embodiments of the present disclosure.

FIGS. 6A-6E illustrate various forms and applications of a wearable device, according to aspects of the present disclosure.

### DETAILED DESCRIPTION

The present disclosure is drawn to devices and methods for regulating temperature, among other aspects. Reference will now be made in detail to aspects of the present disclosure, examples of which are further discussed below.

The terminology used below may be interpreted in its broadest reasonable manner, even though it is being used in conjunction with a detailed description of certain specific examples of the present disclosure. Indeed, certain terms may even be emphasized below; however, any terminology intended to be interpreted in any restricted manner will be overtly and specifically defined as such in this Detailed Description section. Both the foregoing general description and the following detailed description are exemplary and explanatory only and are not restrictive of the features, as claimed.

As used herein, the terms “comprises,” “comprising,” “having,” “including,” or other variations thereof, are intended to cover a non-exclusive inclusion such that a process, method, article, or apparatus that comprises a list of elements does not include only those elements, but may include other elements not expressly listed or inherent to such a process, method, article, or apparatus. In this disclosure, relative terms, such as, for example, “about,” “substantially,” “generally,” and “approximately” are used to indicate a possible variation of ±10% in a stated value or characteristic. Additionally, the term “exemplary” as used herein is used in the sense of “example,” rather than “ideal.”

An exemplary wearable device, also referred to herein as a wearable, may be any device in the form of, or incorporated into, various objects, such as clothing, accessories, and/or consumer products, that allows for said wearable device to be in contact with the human body. Additionally, the wearable device may be configured for determining and/or regulating one or more features of a subject (e.g., a temperature).

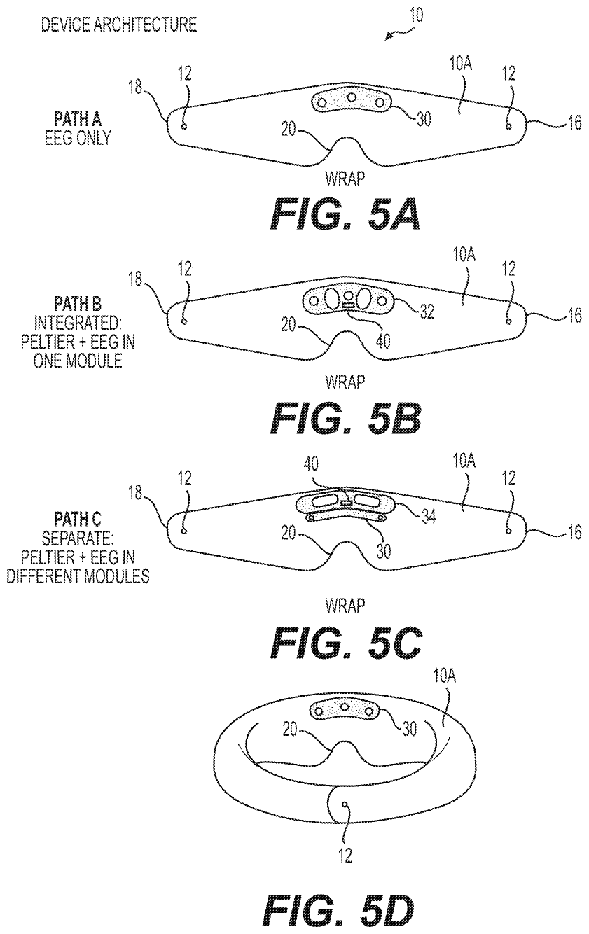

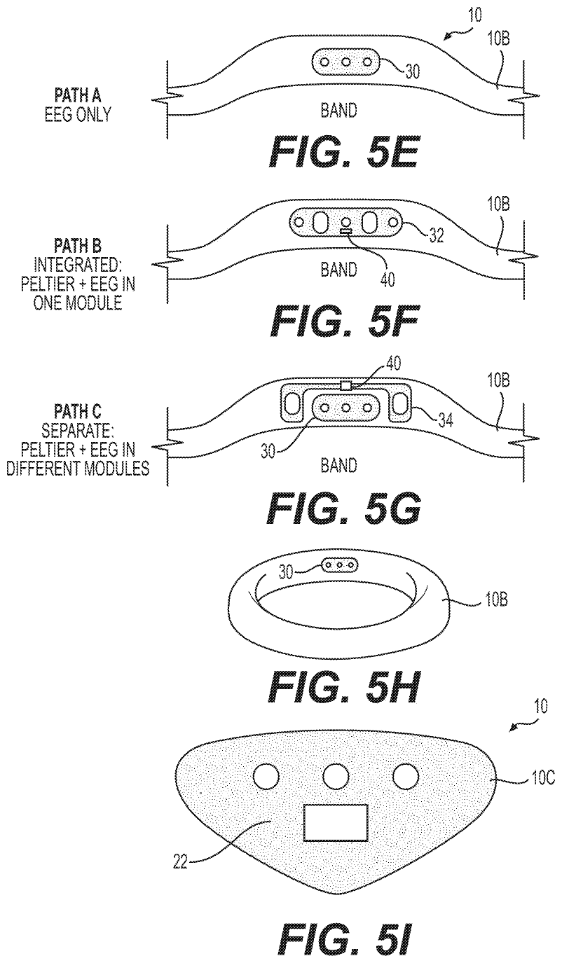

An exemplary wearable device may have any appropriate shape and/or size. For example, an intended application of the wearable device may dictate a suitable shape and/or size of the wearable device. Referring to FIGS. 5A-5I, exemplary wearable devices according to the present disclosure may include a base 10. Base 10 may comprise any suitable structure configured to support one or more electrodes and/or a Peltier effect module, as will be described in further detail below. Base 10 may comprise an elastic, stretchable, or otherwise conformable strip of material (e.g., fabric). In some arrangements, base 10 may be comprised of a “breathable,” light-weight, and/or loose-knit fabric so as to avoid excessive perspiration by the subject.

Base 10 may have any appropriate size and/or shape. For example, base 10 may be sized to fit around a head of the subject, an arm of the subject, a leg of the subject, a wrist of the subject, an ankle of the subject, or the like, without departing from the scope of the present disclosure. Optionally, base 10 may be selectively sizeable. That is, base 10 may include a size adjustment structure (e.g., a D-ring structure, a slide structure, etc.). Additionally or alternatively, base 10 may include a closure structure 12, described in more detail below, that may permit selective sizing of base 10.

In some arrangements, as shown in FIGS. 5A-5D, base 10 may be in the shape of a wrap, e.g., a wrap-shaped base 10A. For example, when in position on the subject (e.g., around a head of the subject) a first portion 16 (e.g., a first end) of wrap-shaped base 10A may overlap, overlie, and/or contact a second portion 18 (e.g., a second end) of wrap-shaped base 10A (FIG. 5D). As such, the wrap-shaped base 10A may include a closure structure 12 (or closure assembly), e.g., Velcro, a zipper, snaps, buttons, magnets, or the like configured to maintain a position of first portion 16 of wrap-shaped base 10A relative to second portion 18 of wrap-shaped base 10A, or vice versa, thereby allowing the wrap-shaped base 10A to securely close around the body (e.g., head) of the subject. As shown in FIGS. 5A-5D, wrap-shaped base 10A may include one or more features 20 arranged to accommodate the subject. For example, feature 20 may include an opening or notch sized the accommodate a nose of the subject, thereby permitting enhanced comfort of the subject while wearing base 10.

In some arrangements, for example, base 10 may be in the shape of a band, e.g., band-shaped base 10B, as shown in FIGS. 5E-5H. Band-shaped base 10B may differ in size (e.g., diameter), depending on the intended application of the wearable device, and/or may be selectively stretchable/conformable so as to securely attach to a subject (e.g., a head of the subject). For example, band-shaped base 10B may include a loop of material configured to surround a portion of the subject. As such, band-shaped base 10B may not include a closure structure 12. That is, band-shaped base 10B may be free from, or otherwise not include, closure structure 12.

In other arrangements, as shown in FIG. 5I, base 10 may be in the shape of a crest, e.g., a crest-shaped base 10C. In one arrangement, crest-shaped base 10C may have one side carrying a replaceable adhesive 22 configured to adhere to a portion of the body of the subject. For example, one side (e.g., a body-facing side) of crest-shaped base 10C may include an adhesive material 22 thereon for secure attachment to the body of the subject. In some arrangements, a liner or film may cover the adhesive material 22 until application of crest-shaped base 10C to the body of the subject. Crest-shaped base 10C may also differ in size, depending on the intended application of the wearable. For example, crest-shaped base 10C may be sized so that it covers a part of the torso of a subject (e.g., chest, stomach, back, etc.). In another example, crest-shaped base 10C may be sized to fit on the forehead of a subject. Thus, wearable devices of the present disclosure are not particularly limited with respect to shape and size, and may be configured according to an intended application.

In some arrangements, one or more exemplary wearable devices may include an electrode arrangement on a first side, e.g., an inside, a subject-facing, and/or a subject-contacting side, of the wearable device, including at least one electrode. The electrode arrangement may be for monitoring one or more features of a subject, e.g., the wearer of the wearable device, and/or delivering stimulation, e.g., transcranial stimulation to the subject. For example, in some arrangements, an exemplary wearable device may include three electrodes. In alternative arrangements, an exemplary wearable device may include a greater or fewer number of electrodes. In some arrangements, electrodes may be arranged throughout the wearable device. In other arrangements, electrodes may be arranged in a single area of the wearable device, designated for the electrodes. Exemplary arrangements of electrodes are discussed in further detail below, and may include one or more electrodes positioned within, along, or on an electrode module 30 (e.g., FIGS. 5A, 5C, 5E, and 5G), or within, along, or on a combination electrode/Peltier effect module 32 (“combination module 32”) (e.g., FIGS. 5B and 5E).

In some arrangements, an exemplary wearable device may include a thermocouple configured for application of a thermoelectric effect on a first side, e.g., an inside, a subject-facing, and/or a subject-contacting side, of the wearable device. That is, an exemplary wearable device may be arranged for the direct conversion of temperature differences to electric voltage and vice versa via the thermocouple. Upon application of a voltage, the thermocouple may create a temperature difference between the two sides of the wearable device. For example, upon application of a voltage, the first side of the wearable device may be cooled, while a second side of the wearable device, e.g., an outside, a subject non-contacting, and/or subject non-facing side, opposite the first side, may be heated. In other arrangements, upon application of a voltage, the first side of the wearable device may be heated while the second side of the wearable device may be cooled. An applied temperature gradient may cause charge carriers in the material of the wearable device to diffuse from the hot side to the cold side. In some arrangements, at least a portion of the wearable device may include electrically conductive material to facilitate application of the temperature gradient. Such electrically conductive materials may include intrinsically conductive fibers and polymers, e.g., carbon fiber, or base substrates that are coated, printed, and/or embedded with a conductive material, e.g., carbon, nickel, copper, gold, silver, and titanium.

Exemplary wearable devices may be configured for application of a thermoelectric effect according to any one or more of the Seebeck effect, the Peltier effect, the Thomson effect, or other similar effects. Any one or more of the above thermoelectric effects may be used to generate electricity, measure temperature, measure perspiration/humidity, and/or change the temperature of the wearable device.

In some arrangements, the Peltier effect may be employed in exemplary wearable devices, via a thermocouple. As such, the thermocouple may be positioned within, along, or on combination module 32 (e.g., FIGS. 5B and 5F), or Peltier module 34 (e.g., FIGS. 5C and 5G). While the remainder of this disclosure refers to a Peltier module and/or a combination module including a Peltier module, the disclosure is not so limited. Rather, any of the above mentioned effects (e.g., Seebeck effect, the Peltier effect, the Thomson effect) may be used in accordance with the teachings of the present disclosure. The Peltier effect is a phenomenon in which a potential difference, or a voltage, applied across a thermocouple triggers a temperature difference between the junctions of two different conductors or semiconductors in the thermocouple. In other words, when a current is made to flow through a junction between the two different conductors or semiconductors, heat may be added at one junction, e.g., a heating effect, and heat may be removed from the other junction, e.g., a cooling effect. Examples of different conductor materials include, but are not limited to, copper and bismuth. By application of the Peltier effect, a voltage may be applied to a thermocouple to cause a current to pass through the different conductors or semi-conductors, thereby causing a cooling effect and a heating effect on opposite sides of the thermocouple. Furthermore, the polarity of an applied voltage dictates the direction, e.g., the sides of the thermocouple, in which a cooling effect and heating effect take place. Thus, a polarity of the applied voltage may be reversed such that the side of the thermocouple previously providing a cooling effect, now provides a heating effect and vice versa.

As the direction of heating and cooling is determined by the polarity of the applied voltage, upon reversing the polarity of the applied voltage, an exemplary wearable device employing at least one thermocouple configured for application of the Peltier effect (hereinafter “Peltier thermocouple”), can be used as a temperature controller or regulator. That is, for example, during the course of use of the wearable device, a subject may exhibit an elevated temperature above a temperature threshold or a threshold temperature range, and the wearable device may be arranged such that application of a voltage results in cooling of the first side of the wearable device via the Peltier effect. If at some point during use of the wearable device, the subject exhibits a reduced temperature below a threshold or threshold range, a polarity of the applied voltage may be reversed such that application of voltage to the wearable device may result in heating of the first side of the wearable device via the Peltier effect.

More specifically, in an exemplary wearable device, voltage may be applied in response, at least in part, to a dynamically measured temperature or a dynamically measured perspiration/humidity level. Temperature may be measured via a temperature sensor of the wearable device, e.g., at least one electrode of the wearable device, or a separate temperature sensor in direct or indirect communication with the wearable device. For example, upon receipt of a signal from a temperature sensor that a subject has a temperature elevated relative to a threshold temperature or a threshold temperature range, the voltage may be applied to the wearable device such that the first side of the wearable device may be cooled so as to reduce a temperature of the subject (while the second side of the wearable device is heated). Additionally, upon receipt of a signal from a temperature sensor that a subject has a temperature below a temperature threshold or a threshold temperature range, the voltage may be applied to the wearable device such that the first side of the wearable device may be heated so as to increase a temperature of the subject (while the second side of the wearable device is cooled). Further, upon receipt of a signal from a temperature sensor that a subject has a temperature within the threshold temperature range, the voltage applied to the wearable device may be delayed such that the present heating or cooling effect on the first side of the wearable device may be maintained. Additionally or alternatively, a perspiration/humidity level may be measured via a perspiration/humidity sensor of the wearable device, e.g., at least one electrode of the wearable device, or a separate perspiration/humidity sensor in direct or indirect communication with the wearable device. For example, upon receipt of a signal from a perspiration/humidity that a subject has a perspiration/humidity level elevated relative to a threshold perspiration/humidity level or a threshold perspiration/humidity level range, the voltage may be applied to the wearable device such that the first side of the wearable device may be cooled so as to reduce a perspiration/humidity level of the subject (while the second side of the wearable device is heated). Additionally, upon receipt of a signal from a perspiration/humidity level sensor that a subject has a perspiration/humidity level below a temperature threshold or a threshold perspiration/humidity level range, the voltage may be applied to the wearable device such that the first side of the wearable device may be heated so as to increase a perspiration/humidity level of the subject (while the second side of the wearable device is cooled). Further, upon receipt of a signal from a perspiration/humidity level sensor that a subject has a perspiration/humidity level within the threshold temperature range, the voltage applied to the wearable device may be delayed (or otherwise maintained at a present magnitude) such that the present heating or cooling effect on the first side of the wearable device may be maintained.

Voltage may also be applied in response, at least in part, to a change or pattern of brain activity, e.g., as measured via at least one electrode of the wearable device. For example, upon the receipt of a dynamically measured signal indicative of a specified change or pattern of brain activity, e.g., a pattern of brain activity indicative of a subject entering rapid-eye-movement (REM) sleep, being in REM sleep, and/or exiting REM sleep, a voltage may be applied to the wearable device such that the first side of the wearable device may be cooled so as to reduce a temperature or perspiration/humidity of the subject (while the second side of the wearable device is heated). Additionally, upon the receipt of a signal indicative of a specified change or pattern of brain activity, e.g., a pattern of brain activity indicative of a subject entering rapid-eye-movement (REM) sleep, being in REM sleep, and/or exiting REM sleep, a voltage may be applied to the wearable device such that the first side of the wearable device may be heated so as to increase a temperature or perspiration/humidity level of the subject (while the second side of the wearable device is cooled). Adjustments of temperature or perspiration/humidity level of a subject may aid or promote a subject to continue a desired sleep cycle such as, for example, REM sleep. Any of the electrodes of the wearable device may be arranged for measuring EEG brain patterns.

Additionally or alternatively, a subject may manually select a desired temperature according to their own personal comfort. For example, an exemplary wearable device may include one or more actuators 40 (e.g., FIGS. 5B, 5C, 5E, 5G) such as, for example, a dial, a push button, a slider, and the like. The actuator 40 may be manually adjusted via the subject so as to adjust a temperature of the first side of the wearable device. For example, the subject may select between low, medium, and high temperature settings via the actuator 40. That is, a subject may wish the first side of the wearable device to have a low, e.g., cold, temperature, a medium, e.g., body and/or room, temperature, or a high, e.g., elevated above body or room, temperature. While a single actuator with three settings is described, the disclosure is not so limited and other arrangements are contemplated. For example, the actuator 40 of the wearable device may have more than three levels and/or may have a first plurality of settings relating to a cooling of the wearable device and a second plurality of settings relating to a heating of the wearable device. In another example, the subject may select a specific temperature, e.g., 74° F., 20° C., etc., via the actuator 40. To select a specific temperature, the actuator 40 may be in any form suitable for such actuation, for example, a dial. In still further arrangements, a desired temperature setting may be selected via a subject on a separate device. For example, a subject may select a desired temperature setting via an application loaded on a smartphone, tablet, computer, or the like. The separate device, e.g., smartphone, may then wirelessly communicate with the wearable device to adjust the temperature of the wearable device according to the subject's preference.

As noted above, exemplary wearable devices may have a variety of arrangements and configurations of electrodes, electrode modules 30, combination modules 32, and/or Peltier modules 34. In some arrangements, an exemplary wearable device may have one (e.g., only one) relatively large module/electrode, e.g., 40×40 mm, or an array of relatively large modules/electrodes on one side of a wearable device. In other arrangements, an exemplary wearable device may have one relatively small module/electrode, e.g., 10×10 mm, or an array of relatively small modules/electrodes on one side of a wearable device. In other arrangements, an exemplary wearable device may have one medium-sized module/electrode, e.g., 20×20 mm, or an array of medium-sized modules/electrodes on one side of a wearable device. An array of modules/electrodes may be a plurality of modules/electrodes in any arrangement or layout, e.g., a horizontally linear arrangement, and any distribution on an entire surface, or portion thereof, of base 10. Concerning the distribution, a plurality modules/electrodes may be, for example, a plurality of modules/electrodes concentrated in one or multiple areas of base 10 of a wearable device, or, in another example, spread across base 10 of a wearable device. In other arrangements, an exemplary wearable device may further include additional elements on one side of a wearable device, such as any conductive fabrics arranged on the first side of the wearable device.

Additional arrangements of exemplary wearable devices, including one or more of an electrode module 30, combination module 32, or Peltier module 34 are illustrated in FIGS. 5A-I. In some arrangements, as illustrated by FIGS. 5A, 5D, 5E, and 5H, an exemplary wrap-shaped or band-shaped wearable device may have at least one electrode module 30 on the first side of the wearable device. In other arrangements, as illustrated by FIGS. 5B and 5F, an exemplary wrap-shaped or band-shaped wearable device may have at least one combination module 32, including at least one electrode and at least one thermocouple, on the first side of the wearable device. In other arrangements, as illustrated by FIGS. 5C and 5G, an exemplary wrap-shaped or band-shaped wearable device may have at least one electrode module 30 and at least one Peltier module 34, separate (e.g., disparate, distinct) from one another, on the first side of the wearable device. The aforementioned module arrangements illustrated in FIGS. 5A-H may also be applied to exemplary wearable devices in other, different shapes, e.g., a crest-shaped wearable device as illustrated by FIG. 5I. As discussed above, in other arrangements, one side of an exemplary wearable device may further include additional elements, e.g., actuator 40. For example, actuator 40 may be a part of or included on at least one module of the exemplary wearable device, as illustrated in FIGS. 5B-5C and 5F-5G, or may be a separate element from the module(s).

With regard to an application of an exemplary wearable device, the wearable device may include any one or more of a headband, a wrap, adhesive patches, a hat or any head covering (e.g., a ski cap, a ball cap, etc.), a helmet, and a wristband. A wearable device may also include any article of clothing, including a jacket/coat, shirt, pants, an undergarments, socks, gloves, shoes, and boots. Apart from objects that are intended to be worn, an exemplary wearable device may also include an article of bedding, e.g., a blanket, sheets, pillows, and/or any fabric or other material worn or used to cover or have contact with all or part of a body, and other consumer products, e.g., containers, coffee cups/mugs, cup liners, tumblers, earphones/headphones, steering wheels, or seats, such as car seats (e.g., a safety device).

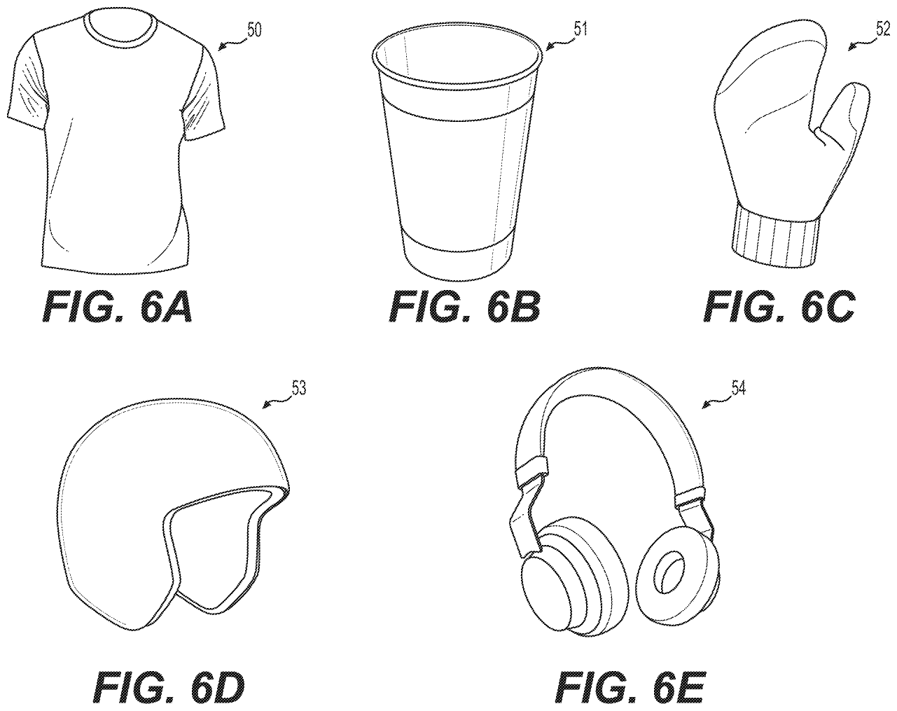

FIGS. 6A-6E illustrate various applications of the exemplary wearable devices, according to this disclosure. The Peltier module(s), combination module(s), and/or electrode module(s) (not shown in FIGS. 6A-E) may be located at any position of any of these exemplary wearable devices, illustrated by FIGS. 6A-E. The exemplary wearable devices of FIGS. 6A-6E may feature dynamic adjustments of cooling or heating based on a determined body temperature of a subject in contact with said exemplary wearable devices, and/or manual actuation of cooling or heating via an actuator including level settings, e.g., low, medium, or high cooling/heating, that may be included on the exemplary wearable device. The form of the actuator is not limited, and it may be a dial, a push button, a slider, and the like.

FIG. 6A depicts an exemplary wearable device in the form of a shirt 50. In one example, a subject wearing shirt 50 may experience a cooling effect when their body temperature is determined to be above a preset temperature range or threshold, or additionally or alternatively, perceived as uncomfortably warm by the subject. Alternatively, a subject wearing said shirt 50 may experience a heating effect when their body temperature is determined to be below a preset temperature range or threshold, or additionally or alternatively, perceived as uncomfortably cool by the subject. In another example, based on a subject's preference, the subject may manually select the level of cooling or heating of shirt 50 via an actuator that may be included on shirt 50.

FIG. 6B depicts an exemplary wearable device applied to a container (e.g., beverage/food container, liquid or substance container, thermos, cooler, etc.), coffee cup/mug, a temperature sleeve slid over a cup/mug, or a tumbler 51. In one example, a subject holding coffee cup/mug, temperature sleeve, or tumbler 51 may experience the outer surface of coffee cup/mug, temperature sleeve, or tumbler 51 cooling when the temperature of their hand is determined to be above a preset temperature range or threshold, or additionally or alternatively, perceived as uncomfortably warm by the subject. Alternatively, a subject holding coffee cup/mug, temperature sleeve, or tumbler 51 may experience the outer surface of coffee cup/mug or tumbler 51 heating when the temperature of their hand is determined to be below a preset temperature range or threshold, or additionally or alternatively, perceived as uncomfortably cool by the subject. Thus, a user may experience coffee cup/mug, temperature sleeve, or tumbler 51 being cool to the touch while carrying hot liquid or a user may experience coffee cup/mug, temperature sleeve, or tumbler 51 being warm to the touch while carrying cool liquid, thereby facilitating maintaining a temperature of a liquid/substance within a container at a desired level while allowing comfortable grasping/holding of the container including the liquid/substance. In other words, a user may selectively choose to warm/heat a substance in a container and/or cool an exterior of the container, or vice versa, depending on user preference. In another example, based on a subject's preference, the subject may manually select the level of cooling or heating of coffee cup/mug, temperature sleeve, or tumbler 51 via an actuator that may be included on coffee cup/mug, temperature sleeve, or tumbler 51.

FIG. 6C depicts an exemplary wearable device in the form of a glove or mitten 52. In one example, a subject wearing glove or mitten 52 may experience a cooling effect in glove or mitten 52 when their hand temperature is determined to be above a preset temperature range or threshold, or additionally or alternatively, perceived as uncomfortably warm by the subject. Alternatively, a subject wearing glove or mitten 52 may experience a heating effect in glove or mitten 52 when their hand temperature is determined to be below a preset temperature range or threshold, or additionally or alternatively, perceived as uncomfortably cool by the subject. In another example, based on a subject's preference, the subject may manually select the level of cooling or heating within glove or mitten 52 via an actuator that may be included on glove or mitten 52.

FIG. 6D depicts an exemplary wearable device in the form of a helmet 53. In one example, a subject wearing helmet 53 may experience a cooling effect in helmet 53 when their head temperature is determined to be above a preset temperature range or threshold, or additionally or alternatively, perceived as uncomfortably warm by the subject. Alternatively, a subject wearing helmet 53 may experience a heating effect in helmet 53 when their head temperature is determined to be below a preset temperature range or threshold, or additionally or alternatively, perceived as uncomfortably cool by the subject. In another example, based on a subject's preference, the subject may manually select the level of cooling or heating within helmet 53 via an actuator that may be included on helmet 53. It is noted that, while the exemplary wearable device of FIG. 6D is in the form of helmet 53, exemplary wearable devices may be in the form of other head coverings, e.g., a hat, ski cap, and the like, as well.

FIG. 6E depicts an exemplary wearable device in the form of headphones 54. In on example, a subject wearing headphones 54 may experience a cooling effect from headphones 54 when their ear or head temperature is determined to be above a preset temperature range or threshold, or additionally or alternatively, perceived as uncomfortably warm by the subject. Alternatively, a subject wearing headphones 54 may experience a heating effect from headphones 54 when their ear or head temperature is determined to be below a preset temperature range or threshold, or additionally or alternatively, perceived as uncomfortably cool by the subject. In another example, based on a subject's preference, the subject may manually select the level of cooling or heating from headphones 54 via an actuator that may be included on headphones 54.

Any one or more of the described wearable devices may be arranged for connection to a charging base. For example, any one or more of the described wearables may include a battery requiring recharging periodically. Such a charging base may be arranged for either wired or wireless charging. For example, the charging base may include a power or charging cord extending between the charging base and the wearable device such that the wearable device may be plugged in, e.g., physically connected, to the charging base. Alternatively, the charging base may be arranged for inductive charging of the battery of the wearable device without the use of a direct plug connection between the wearable device and the charging base.

In either arrangement (wired or wireless charging), the disclosed charging base may include one or more light sources, e.g., lamps, lights, LEDs, or the like. Such a light source may be arranged to illuminate when pre-determined criteria are met. For example, the charging base may illuminate at a certain chronological time. For example, the charging base may comprise an alarm clock which may flash or otherwise illuminate at a specified time so as to wake a subject. In some arrangements, the charging device may illuminate dynamically or in response to a signal. That is, the charging base may illuminate according to a sensed sleep state of a subject using the wearable device. For example, any one or more of the disclosed electrodes of the wearable device may sense EEG brain patterns. The wearable device may communicate the detected EEG brain patterns with the charging base via any appropriate manner, e.g., wireless, wired, BlueTooth, etc. For example, upon the detection of a subject exhibiting REM sleep and the delivery of a signal to the charging base indicative of the detected REM sleep, the charging base may illuminate according to a specified pattern or color. That is, for each stage, e.g., deep, light, REM, etc., of a subject's sleep cycle, the charging base may illuminate according to a unique, corresponding, specified pattern or color. By way of example only, when it is determined that the subject is in a stage of deep sleep, light sleep, or REM, the charging base may illuminate blue, purple, and pink, respectively. As such, others in a room may quickly determine which stage of sleep the subject is in merely by observing the color or pattern of lights of the charging base. As such, another may avoid waking the subject during REM sleep.

Additionally, multiple charging docks within a specific group may be synced, e.g., wirelessly synced, with one another. For example, all charging bases within a single home, e.g., associated with one or more members of a household, may be synched. By way of example only, each charging base associated with one or more parents and children or relatives may be synched. Additionally, multiple charging bases across different households/locations may be synched. For example, each charging base of a group of people may be synched. By way of example only, the charging bases of exercise partners may be synced so as to coordinate the group. As such, an alarm to wake any member of the group of people may be delayed so as to coordinate wake up times until all members are ready to wake. In some arrangements, one or more alarms of the group of members may be delayed based on an additional factor. For example, if it is determined that it is raining, snowing, or otherwise not ideal for exercise, one or all of the alarms of the group may be cancelled to avoid interrupting their sleep.

Any of the described charging bases may additionally or alternatively emit a sound. For example, in addition to a blinking light or a constant bright light to wake a subject, the charging base may include an audible alarm to assist in waking the subject. Alternatively or additionally, the charging base may emit one or more soothing or subject-selected sounds to promote sleep. Further, any of the described charging bases may take the form of a phone charger or an alarm clock, with or without connection to any of the disclosed wearable devices. For example, a mobile phone charging base may be arranged to illuminate at a pre-determined time so as to wake a subject. Additionally, an alarm clock may be arranged to illuminate at a pre-determined time so as to wake a subject.

While principles of the present disclosure are described herein with reference to illustrative embodiments for particular applications, it should be understood that the disclosure is not limited thereto. Those having ordinary skill in the art and access to the teachings provided herein will recognize additional modifications, applications, embodiments, and substitution of equivalents all fall within the scope of the embodiments described herein. For example, in at least some aspects, the disclosed embodiments may not include an electrode or electrode module for sensing a temperature, humidity/perspiration, or EEG pattern of a subject. In such a manner, any of the described arrangements may be actuated only manually by an actuator (e.g., actuator 40) so as to adjust the temperature according to user preference. Accordingly, the invention is not to be considered as limited by the foregoing description.

### Testing Wearable Devices Employing the Peltier Effect

Various tests have been conducted to assess the effects the disclosed, exemplary wearable devices may have on participants. For example, two participants were subjected to a series of perception tests, to help gauge how warm or cool each participant felt in varying conditions, e.g., while not wearing a wearable device and while wearing a wearable device.

### Perception Test: Control Benchmark

As a perception test control benchmark, both Participant 1 and Participant 2 wore headbands that had no Peltier effect inducing capabilities. The perceived warmth/coolness over time of Participants 1 and 2 was recorded.

### Second Perception Test

In a second perception test, both Participant 1 and Participant 2 wore a wearable device employing two Peltier thermocouples of 20×40 mm, on the first side of the wearable device. The wearable device, while powered on, applied 0.3 mAh without any modulation. The perceived warmth/coolness of Participants 1 and 2 was recorded until the wearable device was powered off.

### Third Perception Test

In a third perception test, both Participant 1 and Participant 2 wore a wearable device employing two Peltier thermocouples of 20×40 mm, on the first side of the wearable device. The wearable device, while powered on, applied 0.3 mAh cycling in 1 minute intervals. The perceived warmth/coolness of Participants 1 and 2 was recorded until the wearable device was powered off.

### Measuring the Forehead Temperature of Participant 1

In addition to the perception tests, the forehead temperature of Participant 1 was measured to observe the changes or fluctuations in forehead temperature, as a result of the varying conditions Participant 1 was subjected to during the perception tests.

### Results of the Testing

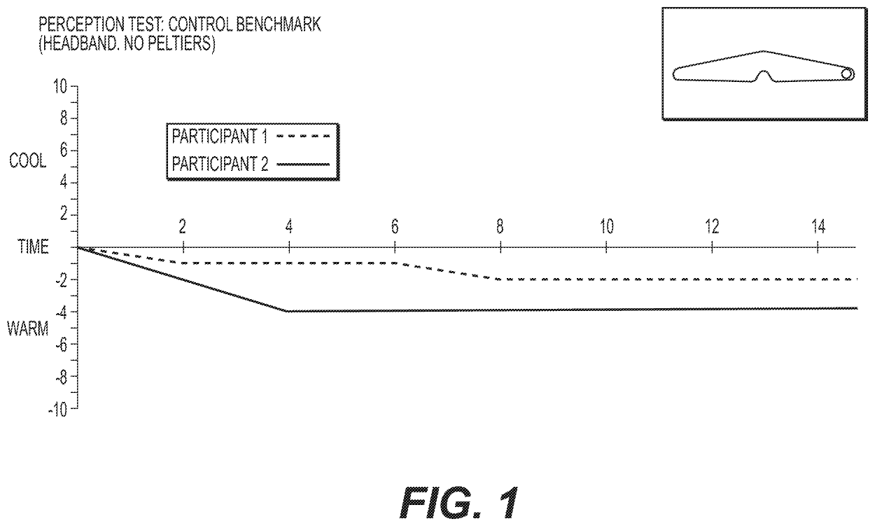

FIGS. 1-4 illustrate, through a series of charts, the results of the perception tests and forehead temperature measurement, discussed above.

FIG. 1 is a chart illustrating the perceived warmth/coolness of the two participants during the first perception test setting the control benchmark. As can be seen in FIG. 1, both participants felt increasingly warmer after a short duration of time (e.g., 4-8 minutes), and remained warm over the remaining duration of time.

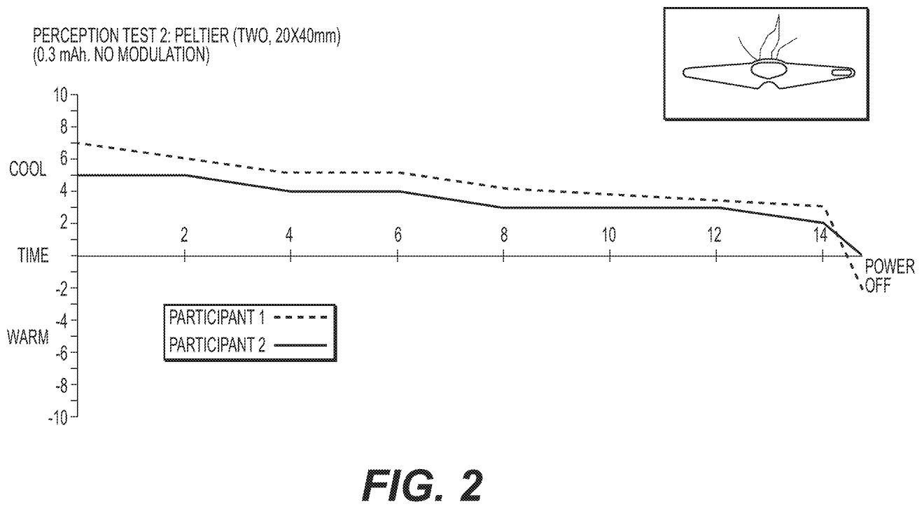

FIG. 2 is a chart illustrating the perceived warmth/coolness of the two participants during the second perception test, in which the participants wore wearable devices employing the Peltier effect, over time. As can be seen in FIG. 2, both participants felt significantly cooler in comparison to the first perception test, in which both participants wore headbands that had no Peltier effect inducing capabilities. FIG. 2 also shows that both participants remained feeling cool until the wearable devices were powered off.

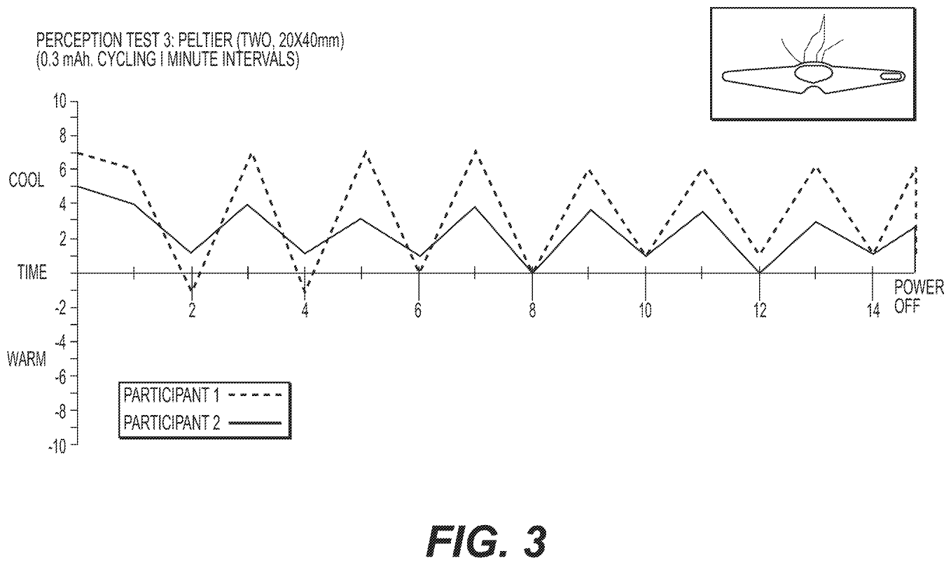

FIG. 3 is a chart illustrating the perceived warmth/coolness of the two participants during the third perception test, in which the participants wore wearable devices cycling the Peltier effect in 1 minute intervals, over time. As can be seen in FIG. 3, both participants felt significantly cooler during the periods of time in which the Peltier effect of the wearable device was cycling on, in comparison to the periods of time in which the Peltier effect was cycling off. This remained the case until both wearable devices were powered off.

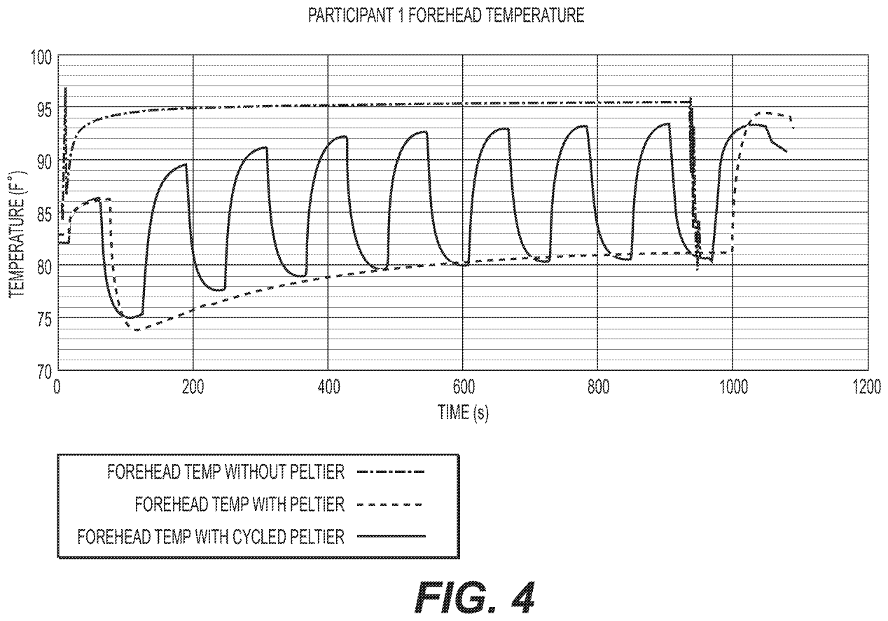

FIG. 4 is a chart illustrating the forehead temperatures of Participant 1 during all three perception tests. As can be seen, the forehead temperatures correlate with the perceived warmness/coolness of Participant 1 during the perception tests. During the first perception test setting the control benchmark, Participant 1 had a forehead temperature of around 95° F., without any application of the Peltier effect. During the second perception test, Participant 1 had a forehead temperature ranging from 74° F. to 81° F., while the wearable device employing the Peltier effect was powered on. During the third perception test, Participant 1 had a forehead temperature fluctuating from as low as 75° F. to as high as 92° F., while the wearable device cycled the Peltier effect in 1 minute intervals. Thus, FIGS. 1-4 demonstrate the ability of an exemplary wearable device, employing the Peltier effect, to regulate and control the temperature of a subject wearing said wearable device.

What is claimed is:

1. A wearable, comprising:
a first side;
a second side;
at least one electrode; and
at least one thermocouple,
wherein the first side is a side in contact with a subject and the second side is a side that is opposite of the first side, wherein the at least one electrode and the at least one thermocouple are arranged on the first side.

2. The wearable of claim 1, wherein the thermocouple is configured for application of the Peltier effect, thereby cooling or heating the first side of the wearable.

3. The wearable of claim 1, further comprising:
an actuator, wherein subject manipulation of the actuator is configured to adjust a temperature of the first side of the wearable and/or a level of relative cooling or heating.

4. The wearable of claim 3, wherein the actuator includes at least one of a dial, a push button, or a slider.

5. The wearable of claim 1, wherein the wearable includes at least one of a band, a wrap, or an adhesive crest.

6. The wearable of claim 5, wherein the wearable includes the wrap, and wherein the wrap includes at least one closure structure.

7. The wearable of claim 1, wherein the wearable includes one or more of an article of clothing, an article of bedding, or a consumer product.

8. The wearable of claim 1, further comprising:
an electrode module; and
a thermocouple module,
wherein the electrode module includes the at least one electrode, the thermocouple module includes the at least one thermocouple, and the electrode module and the thermocouple module are arranged on the first side.

9. The wearable of claim 1, further comprising:
a combination module, wherein the combination module includes the at least one electrode and the at least one thermocouple, and the combination module is arranged on the first side.

10. The wearable of claim 1, wherein the at least one electrode is configured to measure temperature or perspiration/humidity.

11. The wearable of claim 1, wherein the at least one electrode is configured to measure EEG brain patterns.

12. A method of dynamically adjusting a temperature of a wearable, comprising:
monitoring a temperature level of a subject via at least one electrode of the wearable; and
based on the monitored temperature level of the subject, applying a voltage across a thermocouple of the wearable resulting in a cooling effect or a heating effect of a first side of the wearable via the Peltier effect;
wherein the first side of the wearable is a side in contact with the subject.

13. The method of claim 12, further comprising:
comparing the monitored temperature level to a preset temperature range; and
based on the monitored temperature level being above the preset temperature range, applying the voltage across the thermocouple resulting in the cooling effect of the first side of the wearable via the Peltier effect.

14. The method of claim 12, further comprising:
based on the monitored temperature level being below the preset temperature range, applying the voltage across the thermocouple resulting in the heating effect of the first side of the wearable via the Peltier effect.

15. The method of claim 12, further comprising:
based on the monitored temperature level being within the preset temperature range, delaying the application of the voltage across the thermocouple.

16. The method of claim 12, further comprising:
reversing a polarity of the applied voltage to the at least one thermocouple resulting in the first side changing from the heating effect to the cooling effect or from the cooling effect to the heating effect.

17. The method of claim 12, further comprising:
monitoring EEG brain patterns of a subject via at least one additional electrode of the wearable; and
based on the monitored EEG brain patterns, applying the voltage across the thermocouple of the wearable resulting in the cooling effect or the heating effect of the first side of the wearable via the Peltier effect.

18. A method of dynamically adjusting a temperature, comprising:
positioning at least one of a wearable or a consumer product relative to a subject;
based on a monitored or perceived condition of the subject, applying a voltage across at least one thermocouple of the at least one of the wearable or consumer product resulting in a heating effect or a cooling effect of a first side of the at least one of the wearable or the consumer product via the Peltier effect,
wherein the first side is a side in contact with the subject.

19. The method of claim 18, further comprising:
reversing a polarity of the applied voltage to the at least one thermocouple resulting in the first side changing from the heating effect to the cooling effect or from the cooling effect to the heating effect.

20. The method of claim 18, wherein the at least one of the wearable or the consumer product includes an article of clothing, footwear, an article of bedding, a head covering, earphones, a container, a temperature sleeve of a container, a safety device, a seat, or a steering wheel.
